(19) United States
(12) Patent Application Publication
(10) Pub. No.: US 2008/0280335 A1
(43) Pub. Date: Nov. 13, 2008

(54) Manufacturing Method of Kaempferol

(75) Inventors: Myeong Hoon Yeom, Gyonggi-do (KR); Jun-seong Park, Gyonggi-do (KR); Won-Seok Park, Seoul (KR); Kyungmi Joo, Gyonggi-do (KR); Ho Sik Rho, Gyonggi-do (KR); Duck Hee Kim, Seoul (KR); Ih Seop Jang, Gyonggi-do (KR); Ok-Sub Lee, Gyonggi-do (KR)

Correspondence Address: MERCHANT & GOULD PC, P.O. BOX 2903, MINNEAPOLIS, MN, 55402-0903, US

(73) Assignee: AMOREPACIFIC CORPORATION, SEOUL (KR)

(21) Appl. No.: 11/795,343

(22) PCT Filed: May 30, 2005
(86) PCT No.: PCT/KR2005/001597

§ 371 Date: Jan. 30, 2008

(30) Foreign Application Priority Data
Jan. 18, 2005 (KR) 10-2005-0004715

(51) Int. Cl. C12P 17/06; C07D 311/04

(52) U.S. Cl. 435/125; 549/400

(57) ABSTRACT

Disclosed is a kaempferol preparing method comprising isolating kaempferol from kaempferol glucosides using an acid, a base, an enzyme or a microbe producing the enzyme. More specifically, the method comprises obtaining a plant extract containing kaempferol glycosides from a plant using water or an organic solvent; and hydrolyzing the plant extract using an acid, a base, an enzyme or a microbe producing the enzyme to isolate kaempferol. The kaempferol glycosides comprise camelliaside A or camelliaside B. The plant extract is derived from a seed or leaf of green tea. When using the method of the invention, it is possible to mass-produce kaempferol, which is one of main physiological activating ingredients, from a plant, particularly a seed or leaf of green tea.

### TECHNICAL FIELD

The present invention relates to a manufacturing method of kaempferol wherein kaempferol is isolated from kaempferol glycosides using an acid, a base, an enzyme or a microbe producing the enzyme.

### BACKGROUND ART

Kaempferol having a following chemical formula 1 is one of representative ingredients of flavonol which is one of flavonoids, and widely distributed in a flower or leaf of a plant.

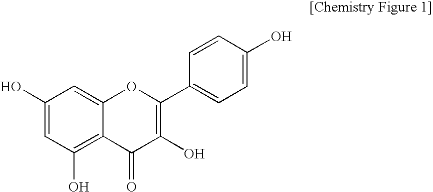

100 or more of types of flavonols have been already known and it is known that kaempferol, quercetin and myricetin among them exist most.

In particular, kaempferol is a substance having excellent physiological activities such as anti-oxidation and anti-inflammatory activities. Accordingly, researches on the various efficacies of kaempferol have been performed and kaempferol was applied to diverse fields. However, since kaempferol, which is currently used, is mostly a plant extract which contains it in an amount of several ppm to several tens ppm only, a substantial efficacy of kaempferol is difficult to be revealed. In addition, since it is difficult to find a plant containing a large quantity of kaempferol and there are no economical merits of isolation and purification for preparing a large quantity of kaempferol, researches on a mass production of kaempferol has seldom been carried out.

Green tea is a beverage having the oldest history in the world. As a concern about the green tea increases in recent years, there have been many researches on ingredients and pharmacological efficacies of the tea. The green tea contains a large amount of threamines and polyphenols, compared to other foods. It is known that a functional ingredient of the green tea is flavan-3-ol-based catechin belonging to multifoliate polyphenols and main ingredients thereof are (+)-catechin, (−)-epicatechin, (−)-epigallocatechin-3-gallate, and (−)-gallocatechin, etc. In particular, it is reported that polyphenols contained in the green tea lowers the level of cholesterol in blood and have anti-oxidization, anti-cancer, detoxification, antibacterial function, tooth decay prevention, aging suppression actions, a whitening effect and a fragrance ingredient, etc. It is also reported that polyphenols contained in the green tea prevent gout, suppresses lipid peroxide and a production of neutral lipid and delays the aging, thereby preventing obesity and improving resisting power of a capillary vessel.

However, the green tea having the various efficacies is mostly used in a form of leaf and a green tea seed containing similar effective ingredients is not used besides a cultivation purpose. In addition, all concerns and researches are focused on catechins of the green tea, especially epigallocatechin gallate (EGCG).

### DISCLOSURE

### Technical Problem

The inventors discovered that a green tea seed, which is not used for a specific purpose, and a leaf of the green tea, which is focused on EGCG, contain a large quantity of kaempferol glycosides having a kaempferol mother nucleus, in particular, glycosides such as camelliaside A and camelliaside B having three sugars attached to kaempferol. From this discovery, the inventors accomplished a method for mass-producing an aglycone type of kaempferol having an excellent physiological activity.

Accordingly, the object of the present invention is to provide a method of isolating and preparing kaempferol from kaempferol glycosides, using an acid, a base, an enzyme or a microbe producing the enzyme. In other words, the object of the invention is to provide a method of isolating and preparing kaempferol from kaempferol glycosides abundantly contained in a green tea seed or leaf, in particular glycosides such as camelliaside A and camelliaside B, etc.

### Technical Solution

In order to accomplish the object, there is provided a kaempferol preparing method comprising isolating kaempferol from kaempferol using an acid, a base, an enzyme or a microbe producing the enzyme.

More specifically, the kaempferol preparing method comprises a first step of obtaining a plant extract containing kaempferol glycosides from a plant, using water or an organic solvent; and a second step of hydrolyzing the plant extract using an acid, a base, an enzyme or a microbe producing the enzyme to isolate kaempferol.

The kaempferol glycosides comprise camelliaside A or camelliaside B.

According to an embodiment of the invention, in the first step, the plant extract may be derived from a green tea seed or leaf.

In addition, according to an embodiment of the invention, the organic solvent may be at least one selected from a group consisting of ethanol, methanol, butanol, ether, ethylacetate and chloroform, or a mixture solvent of the organic solvents and water, preferably 80% ethanol.

According to an embodiment of the invention, the acid may be at least one selected from a group consisting of hydrochloric acid, sulfuric acid and nitric acid, or a mixture solvent of the acids and at least one alcohol selected from a group consisting of ethanol, methanol and butanol. At this time, according to an embodiment of the invention, preferable concentration range of the acid may be 0.1N˜2N, an alcohol content of the mixture solvent may be 10˜50%, a reaction temperature may be 50˜100° C. and a reaction time may be 0.5˜8 hours.

According to an embodiment of the invention, the base may be at least one selected from a group consisting of sodium hydroxide and potassium hydroxide, or a mixture solvent of the bases and at least one alcohol selected from a group consisting of ethanol, methanol and butanol. At this time, according to an embodiment of the invention, a concentration of the base capable of being used may be 0.1N˜2N, an alcohol content of the mixture solvent may be 10˜50%, a reaction temperature may be 50˜100° C. and a reaction time may be 0.5˜24 hours.

According to an embodiment of the invention, the enzyme or the microbe producing the enzyme may be an enzyme decomposing a sugar bond or a microbe producing the enzyme decomposing the sugar bond, and the enzyme may remove the sugar part from the kaempferol glycosides to isolate kaempferol. The kaempferol glycosides may preferably comprise camelliaside A or camelliaside B.

Additionally, according to an embodiment of the invention, more preferably, the enzyme may be at least one selected from a group consisting of glucosidase, arabinosidase, rhamnosidase, xylosidase, cellulase, hesperidinase, naringinase, glucuronidase, pectinase, galactosidase and amyloglucosidase.

Further, according to an embodiment of the invention, the microbe producing the enzyme may be at least one selected from a group consisting of aspergillus sp., bacillus sp., penicillium sp., rhizopus sp., rhizomucor sp., talaromyces sp., bifidobacterium sp., mortierella sp., cryptococcus sp. and microbacterium sp.

### ADVANTAGEOUS EFFECTS

According to the invention, when using a method of isolating kaempferol from kaempferol glycosides with the acid, base, enzyme or microbe producing the enzyme, it is possible to obtain plant extracts containing kaempferol glycosides, particularly camelliaside A or camelliaside B from a plant, particularly a seed or leaf of green tea, and then to mass-produce kaempferol, which is one of main physiological active ingredients, through a hydrolysis method using the acid, base, enzyme or microbe producing the enzyme.

### DESCRIPTION OF DRAWINGS

FIGS. 1 and 2 show results of high speed liquid chromatography measurement measuring changes before and after hydrolyzing using an enzyme an extract of green tea seed of the Example 1 according to a method of the Example 3, wherein FIG. 1 is a view showing contents of camelliaside A and camelliaside B in the extract of green tea before the hydrolysis and FIG. 2 is a view showing a content of kaempferol in the extract of green tea after the hydrolysis.

### BEST MODE

A kaempferol preparing method according to the invention comprises a first step of obtaining a plant extract containing kaempferol glycosides from a plant using water or an organic solvent; and a second step of hydrolyzing the plant extract using an acid, a base, an enzyme or a microbe producing the enzyme to isolate kaempferol.

In the first step, in order to obtain the plant extract containing camelliaside A or camelliaside B, which are kaempferol glycosides, using the water or organic solvent, about one or six times, preferably about three times water or at least one organic solvent selected from a group consisting of ethanol, methanol, butanol, ether, ethylacetate and chloroform, or a mixture solvent of the organic solvents and water is poured to the plant. Then, the plant is extracted while being stirred one to five times at a room temperature to remove fat. About one to eight times, preferably about four times water or the organic solvent is poured to the fat-removed plant. The mixture is extracted under reflux one to five times, and deposited at 10 to 20° C. for one to three days. After that, residues and filtrate are separated through filtration and centrifugation, and extracts obtained by concentrating under reduced pressure the separated filtrate are suspended in water and pigments thereof are removed using ether, etc. Then, water layer is removed one to five times using butanol, etc. After that, an extract is obtained by concentrating under reduced pressure the obtained organic solvent layer, and dissolved in a small quantity of methanol, etc. Then, precipitates produced by adding a large quantity of ethylacetate, etc. to the mixture are dried to obtain the plant extract of the invention.

In the second step, the plant extract is hydrolyzed using an acid, a base, an enzyme or a microbe producing the enzyme to prepare kaempferol.

At this time, when an acid is used, 0.1N˜2N, preferably 1N of an acid or a mixture solvent of the acid and alcohol (preferably, 50% ethanol mixture solvent) is added to the plant extract and then hydrolyzed by heating under reflux in a water bath at 50˜100° C., preferably 80° C. for 1 to 48 hours, preferably 8 hours, thereby providing a reaction solution.

When a base is used, the plant extract is dissolved, then added with 0.1N˜2N, preferably 1N of a base or a mixture solvent of a base and alcohol (preferably, 50% butanol mixture solvent) and hydrolyzed by heating under reflux in a water bath at 50˜100° C., preferably 100° C. for 1 to 48 hours, preferably 8 hours, thereby providing a reaction solution.

When an enzyme is used, the plant extract is dissolved in 5 to 20 times, preferably about 10 times acid buffer solution, added with an enzyme and then stirred in a water bath at about 37° C. for 40 to 55 hours, preferably 48 hours. At the same time, a removal rate of substrate is checked with a thin layer chromatography. When the substrate is completely removed, the mixture is heated in a hot water (80˜100° C.) for 5 to 15 minutes to terminate the hydrolysis reaction, thereby providing a reaction solution.

When a microbe producing the enzyme is used, the plant extract is dissolved in 5 to 10 times, preferably about 10 times ionic water, sterilized at about 121° C. for 30 minutes and cooled to about 30° C. After that, the mixture is inoculated with 5˜10%, based on a liquid amount, microbes cultured in advance, and then cultured at 30° C. for 2 to 5 days, preferably 5 days. A removal rate of substrate is checked with a thin layer chromatography. When the substrate is completely removed, the hydrolysis reaction is terminated and precipitates recovered by centrifugation the culture solution at 5,000 to 10,000 rpm are cleaned three times with distilled water and then centrifuged, thereby providing a reaction solution as precipitates.

As described above, the hydrolysis reaction is performed using an acid, a base, an enzyme or a microbe producing the enzyme. Then, the reaction solution obtained is concentrated under reflux pressure to remove the solvent and alcohol is added to the residues and then the mixture is stirred one to five times. Precipitated salts are removed through a filtration, and filtered filtrate is concentrated under reduced pressure to obtain crude products. The obtained crude products are purified with a silica gel column chromatography (chloroform: methanol=8:1˜4:1), thereby providing kaempferol.

### Mode for Invention

Hereinafter, the invention will be more specifically described with examples and experimental examples. However, it should be noted that the invention is not limited to them.

### EXAMPLE 1

### Preparation of Extract of a Green Tea Seed

61 of hexane was added to 2 kg of green tea seeds and then the mixture was stirred three times at a room temperature to remove fat. 41 of 80% methanol was poured to 1 kg of the fat-removed seeds, the mixture was extracted under reflux three times and then deposited at 15° C. for a day. After that, residues and filtrate were separated through filtration of filter cloth and centrifugation and an extract obtained by concentrating under reduced pressure the separated filtrate was suspended in water and then extracted five times with 11 of ether to remove pigments. Water layer was extracted three times with 500 ml 1-butanol. All 1-butanol layer obtained was concentrated under reduced pressure to obtain 1-butanol extract. The obtained extract was dissolved in a small quantity of methanol and then added to a large quantity of ethylacetate, thereby obtaining precipitates. The produced precipitates were dried, thereby obtaining 250 g of extract of green tea seeds.

### EXAMPLE 2

### Preparation of Extract of a Green Tea Leaf

61 of hexane was added to 2 kg of green tea leaves and then the mixture was stirred three times at a room temperature to remove fat. 41 of 80% methanol was poured to 1 kg of the fat-removed leaves, the mixture was extracted under reflux three times and then precipitated at 15° C. for a day. After that, residues and filtrate were separated through filtration of filter cloth and centrifugation and an extract obtained by concentrating under reduced pressure the separated filtrate was suspended in water and then extracted five times with 11 of ether to remove pigments. Water layer was extracted three times with 500 ml 1-butanol. All 1-butanol layer obtained was concentrated under reduced pressure to obtain 1-butanol extract. The obtained extract was dissolved in a small amount of methanol and then added to a large quantity of ethylacetate. The produced precipitates were dried, thereby obtaining 150 g of extract of green tea leaves.

### EXAMPLE 3

### Preparation of Kaempferol with an Acid Hydrolysis Method

10 g of the extract of green tea seeds, which was obtained from the Example 1, was added to 20 times (v/w) 1N HCl-50% methanol solution (v/v) and then subject to a reflux-heating in a water bath at 80° C. for 8 hours, thereby hydrolyzing sugars bonded to camelliaside A and camelliaside B. After that, the reaction solution was concentrated under reduced pressure to remove the solvent, and ethanol (200 ml) was added to the residues and the mixture was stirred (three times). The resulting precipitated salts were removed through filtration, and filtered filtrate was concentrated under reduced pressure to obtain crude products. The obtained crude products were purified with a silica gel column chromatography (chloroform: methanol=8:1˜4:1), thereby obtaining 0.95 g of kaempferol.

### EXAMPLE 4

### Preparation of Kaempferol with a Base Hydrolysis Method

10 g of the extract of green tea seeds, which was obtained from the Example 1, was dissolved in dry pyridine (500 ml), added to sodium methoxide (powder, 10 g) and was then subject to a reflux-heating in a water bath for 8 hours, thereby hydrolyzing sugars bonded to camelliaside A and camelliaside B. After that, the reaction solution was concentrated under reduced pressure to remove the solvent, and ethanol (200 ml) was added to the residues and the mixture was stirred (three times). The resulting precipitated salts were removed through filtration. Filtered filtrate was concentrated under reduced pressure to obtain crude products. The obtained crude products were purified with a silica gel column chromatography (chloroform: methanol=8:1˜4:1), thereby obtaining 0.25 g of kaempferol.

### EXAMPLE 5

### Preparation of Kaempferol with an Enzyme Digestion Method

10 g of the extract of green tea seeds, which was obtained from the example 1, was dissolved in 100 ml of 0.1 M acetic acid buffer solution (pH 4.5). 2.5 g of enzyme (hesperidinase 0.5 g, naringinase 0.5 g, cellulase 0.5 g, β-glucuronidase 0.2 g, β-galactosidase 0.5 g, amyloglucosidase 0.3 g; manufactured from Sigma company) was added to the mixture and the mixture was periodically checked with a thin layer chromatography while being stirred in a water bath at 37° C. for 48 hours. When the substrate (camelliaside A and camelliaside B) was completely removed, the mixture was heated in hot water (80˜100° C.) for 10 minutes to terminate the reaction. After that, the reaction solution was concentrated under reduced pressure to remove the solvent, and the residues were added to ethanol (200 ml) and stirred (three times). The resulting precipitates were removed through filtration and filtered filtrate was then concentrated under reduced pressure to obtain crude products. The obtained crude products were purified with a silica gel column chromatography (chloroform: methanol=8:1˜4:1), thereby obtaining 1.02 g of kaempferol.

### EXAMPLE 6

### Preparation of Kaempferol Using Microbes

10 g of the extract of green tea leaves, which was obtained from the Example 2, was dissolved in 100 ml of ionic water, sterilized at 121° C. for 30 minutes, cooled to 30° C., inoculated with 5˜10%, based on a liquid amount, Aspergillus niger KCCM 11885 cultured in advance and cultivated at 30° C. for 5 days. A removal rate of substrate was checked with a thin layer chromatography. When the substrate was completely removed, the hydrolysis reaction was terminated and precipitates recovered by centrifuging the culture solution at 5,000 to 10,000 rpm were cleaned three times with distilled water and then centrifuged, thereby obtaining a reaction solution as precipitates. ethanol (200 ml) was added to the precipitates and then the mixture was stirred (three times). Then, the precipitates were removed through filtration and filtered filtrate was concentrated under reduced pressure to obtain crude products. The obtained crude products were purified with a silica gel column chromatography (chloroform: methanol=8:1˜4:1), thereby obtaining 0.62 g of kaempferol.

### EXPERIMENTAL EXAMPLE 1

### Identification of Camelliaside A and Camelliaside B

10 g of the extract of green tea seeds, which was obtained from the Example 1, was purified with a silica gel column chromatography (filled with 100 g silica gel). At this time, chloroform and methanol were used as a development solvent and a ratio of chloroform and methanol was increased from 10:1 to 2:1 so as to raise a concentration gradient and thus to obtain a fractionation. From the fractionation, 0.82 g camelliaside A and 1.24 g camelliaside B were obtained. The obtained products were subject to an identification process (Varian Gemini 2000, 300 MHz, Varian company). As a result of that, since the products exhibited characteristics as shown in the following Table 1, they were identified as camelliaside A and camelliaside B.

<physicochemical properties of camelliaside A>

property: light greenish yellow micro crystal

positive FAB-MS: 756.9[M+H]

<physicochemical properties of camelliaside B>

property: light greenish yellow micro crystal

positive FAB-MS: 726.9[M+H]

TABLE 1
¹H-NMR and ¹³C-NMR data of camelliaside A and camelliaside B

| Camelliaside A | | | Camelliaside B | | |
|---|---|---|---|---|---|
| ¹³C | | ¹H | ¹³C | | ¹H |
| kaempferol | | 1.09(3H, d, 6.3 Hz) | kaempferol | | 1.09(3H, d, 6.3 Hz) |
| 2 | 159.247 | 3.2~3.8(16H, m) | 2 | 158.697 | 3.2~3.8(15H, m) |
| 3 | 134.717 | 4.4(1H, d, H1-Rha) | 3 | 134.827 | 4.4(1H, d, H1-Rha) |
| 4 | 179.475 | 4.7(1H, d, H1-Gal) | 4 | 179.425 | 4.7(1H, d, H1-Gal) |
| 5 | 161.467 | 5.3(1H, d, 7.8 Hz, | 5 | 161.363 | 5.3(1H, d, 7.8 Hz, |
| | | H1-Glc) | | | H1-Glc) |
| 6 | 101.122 | 6.17(1H, d, 1.8 Hz, H6) | 6 | 99.919 | 6.17(1H, d, 1.8 Hz, H6) |
| 7 | 163.019 | 6.37(1H, d, 1.8 Hz, H8) | 7 | 163.038 | 6.37(1H, d, 1.8 Hz, H8) |
| 8 | 95.063 | 6.9(2H, d, 9 Hz, H3\grave{ }, | 8 | 94.847 | 6.9(2H, d, 9 Hz, H3\grave{ }, |
| | | 5\grave{ }) | | | 5\grave{ }) |
| 9 | 166.589 | 8.0(2H, d, 8.7 Hz, H2\grave{ }, 6\grave{ }) | 9 | 165.906 | 8.0(2H, d, 8.7 Hz, H2\grave{ }, 6) |
| 10 | 105.565 | | 10 | 105.725 | |
| 1\grave{ } | 122.936 | | 1\grave{ } | 122.924 | |
| 2\grave{ }, 6\grave{ } | 132.365 | | 2\grave{ }, 6\grave{ } | 132.346 | |
| 3\grave{ }, 5\grave{ } | 116.239 | | 3\grave{ }, 5\grave{ } | 116.167 | |
| 4\grave{ } | 158.595 | | 4\grave{ } | 158.473 | |
| Glc | | | Glc | | |
| 1 | 101.122 | | 1 | 100.830 | |
| 2 | 82.048 | | 2 | 81.927 | |
| 3 | 78.281 | | 3 | 78.205 | |
| 4 | 72.073 | | 4 | 71.424 | |
| 5 | 77.818 | | 5 | 76.839 | |
| 6 | 68.226 | | 6 | 68.112 | |
| Rha | | | Rha | | |
| 1 | 102.211 | | 1 | 102.139 | |
| 2 | 72.293 | | 2 | 72.274 | |
| 3 | 73.856 | | 3 | 73.853 | |
| 4 | 71.402 | | 4 | 72.062 | |
| 5 | 69.713 | | 5 | 69.698 | |
| 6 | 17.853 | | 6 | 17.845 | |
| Gal | | | Xyl | | |
| 1 | 104.476 | | 1 | 105.133 | |
| 2 | 75.378 | | 2 | 74.676 | |
| 3 | 76.945 | | 3 | 77.051 | |
| 4 | 71.274 | | 4 | 70.996 | |
| 5 | 77.867 | | 5 | 66.541 | |
| 6 | 62.591 | | | | |

*Glc: glucose, Rha: rhamnose, Gal: galactose, Xyl: xylose

### EXPERIMENTAL EXAMPLE 2

### Identification of Kaempferol

Since the products, which were prepared in the Examples 3 to 6, exhibited characteristics as follows, they were identified as kaempferol (Verian Gemini 2000, 300 MHz, Varian company)

<physicochemical properties of kaempferol>

property: light greenish yellow micro crystal

positive FAB-MS: 287[M+H]+

¹H-NMR: 6.1 (1H, d, 1.8 Hz), 6.3 (11H, d, 1.8 Hz), 6.8 (2H, dd, 9 Hz), 8.0 (2H, dd, 9 Hz)

¹³C-NMR: 94.467, 99.248, 104.518, 116.265, 123.710, 130.649, 137.069, 147.970, 158.200, 160.480, 162.446, 165.519, 177.285

### EXPERIMENTAL EXAMPLE 3

### Changes of Contents of Kaempferol after the Hydrolysis (Enzyme Digestion)

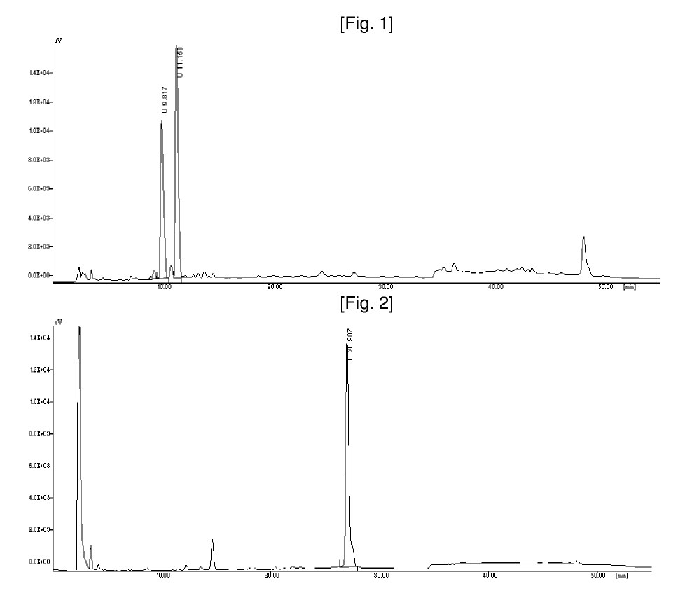

The extract of green tea seeds was prepared according to the Example 1 and then subject to the enzyme digestion reaction as described in the Example 3. After that, a change before and after the enzyme digestion reaction was measured using a high-speed liquid chromatography. At this time, measurement results of contents of camelliaside A and camelliaside B, which were contained in the extract of green tea seeds before the enzyme digestion reaction, were shown in FIG. 1, and measurement results of contents of kaempferol after the enzyme digestion reaction were shown in FIG. 2.

As shown in FIGS. 1 and 2, almost all of camelliaside A and camelliaside B were converted into kaempferol.

### INDUSTRIAL APPLICABILITY

According to the invention, when using a method of isolating kaempferol from kaempferol glycosides with the acid, base, enzyme or microbe producing the enzyme, it is possible to obtain plant extracts containing kaempferol glycosides, particularly camelliaside A or camelliaside B from a plant, particularly a seed or leaf of green tea, and then to mass-produce kaempferol, which is one of main physiological active ingredients, through a hydrolysis method using the acid, base, enzyme or microbe producing the enzyme.

1. A Method for manufacturing kaempferol comprising isolating kaempferol from kaempferol glucosides using an acid, a base, an enzyme or a microbe producing the enzyme.

2. The method according to claim 1, which comprises obtaining a plant extract containing kaempferol glycosides from a plant using water or an organic solvent;
and hydrolyzing the plant extract using an acid, a base, an enzyme or a microbe producing the enzyme to isolate kaempferol.

3. The method according to claim 1, wherein the kaempferol glycosides comprise camelliaside A or camelliaside B.

4. The method according to claim 2, wherein the plant extract is derived from a seed or leaf of green tea.

5. The method according to claim 2, wherein the organic solvent is at least one selected from a group consisting of ethanol, methanol, butanol, ether, ethylacetate and chloroform, or a mixture solvent of the organic solvents and water.

6. The method according to claim 1, wherein the acid is at least one selected from a group consisting of hydrochloric acid, sulfuric acid and nitric acid, or a mixture solvent of the acids and at least one alcohol selected from a group consisting of ethanol, methanol and butanol.

7. The method according to claim 1, wherein the base is at least one selected from a group consisting of sodium hydroxide and potassium hydroxide, or a mixture solvent of the bases and at least one alcohol selected from a group consisting of ethanol, methanol and butanol.

8. The method according to claim 1, wherein the enzyme removes a sugar part from the kaempferol glycosides to isolate kaempferol.

9. The method according to claim 8, wherein the kaempferol glycosides comprise camelliaside A or camelliaside B.

10. The method according to claim 8, wherein the enzyme is at least one selected from a group consisting of glucosidase, arabinosidase, rhamnosidase, xylosidase, cellulase, hesperidinase, naringinase, glucuronidase, pectinase, galactosidase and amyloglucosidase.

11. The method according to claim 1, wherein the microbe producing the enzyme is at least one selected from a group consisting of aspergillus sp., bacillus sp., penicillium sp., rhizopus sp., rhizomucor sp., talaromyces sp., bifidobacterium sp., mortierella sp., cryptococcus sp. and microbacterium sp.

12. The method according to claim 2, wherein the kaempferol glycosides comprise camelliaside A or camelliaside B.

13. The method according to claim 2, wherein the acid is at least one selected from a group consisting of hydrochloric acid, sulfuric acid and nitric acid, or a mixture solvent of the acids and at least one alcohol selected from a group consisting of ethanol, methanol and butanol.

14. The method according to claim 2, wherein the base is at least one selected from a group consisting of sodium hydroxide and potassium hydroxide, or a mixture solvent of the bases and at least one alcohol selected from a group consisting of ethanol, methanol and butanol.

15. The method according to claim 2, wherein the enzyme removes a sugar part from the kaempferol glycosides to isolate kaempferol.

16. The method according to claim 15, wherein the kaempferol glycosides comprise camelliaside A or camelliaside B.

17. The method according to claim 15, wherein the enzyme is at least one selected from a group consisting of glucosidase, arabinosidase, rhamnosidase, xylosidase, cellulase, hesperidinase, naringinase, glucuronidase, pectinase, galactosidase and amyloglucosidase.

18. The method according to claim 2, wherein the microbe producing the enzyme is at least one selected from a group consisting of aspergillus sp., bacillus sp., penicillium sp., rhizopus sp., rhizomucor sp., talaromyces sp., bifidobacterium sp., mortierella sp., cryptococcus sp. and microbacterium sp.
